(19) United States
(12) Plant Patent Application Publication
(10) Pub. No.: US 2015/0223376 P1
(43) Pub. Date: Aug. 6, 2015

(54) Blueberrey plant named 'FF04-14'

(50) ;

(71) Applicant: Fall Creek Farm & Nursery, Inc., Lowell, OR (US)

(72) Inventors: David M. Brazelton, Walterville, OR (US); Adam L. Wagner, Eugene, OR (US); Peter Stefan Boches, Eugene, OR (US); Antonio A. Alamo Bermudo, Seville (ES)

(21) Appl. No.: 14/544,639

(22) Filed: Jan. 27, 2015

Related U.S. Application Data
(60) Provisional application No. 61/965,614, filed on Feb. 3, 2014.

(51) Int. Cl. A01H 5/00

(52) U.S. Cl. PLT/157

(57) ABSTRACT

The new blueberry plant variety ‘FF04-14’ is provided. The variety is produced from a cross of ‘FL00-45’ (female parent, unpatented) by ‘FL97-130’ (pollen parent, unpatented), which can be distinguished by its outstanding features.

### CROSS REFERENCE TO RELATED APPLICATIONS

This application claims the benefit of U.S. Provisional Patent Application Ser. No. 61/965,614 filed on Feb. 3, 2014, which is incorporated herein by reference in its entirety as though fully set forth.

### LATIN NAME OF THE FAMILY, GENUS, AND SPECIES

Family—Ericaceae.

Genus—Vaccinium.

Species—corymbosum hybrid

### VARIETY DENOMINATION

The new blueberry plant claimed is of the variety denominated ‘FF04-14’.

### BACKGROUND OF THE INVENTION

The present invention relates to the discovery of a new and distinct cultivar of southern highbush blueberry known as a Vaccinium corymbosum hybrid referred to as ‘FF04-14,’ as herein described and illustrated.

The new blueberry plant variety ‘FF04-14’ was selected in Lowell, Oreg. in 2006. Plants of ‘FF04-14’ is a commercial variety intended for the hand harvest fresh market. The variety is vigorous, highly branched and leafy and produces a high yield of large fruit. ‘FF04-14’ has an estimated chilling requirement of 300-500 hours, but also produces high fruit yields in an evergreen production system with zero chill hours when the plants are not allowed to enter dormancy.

### PEDIGREE AND HISTORY

The new blueberry plant originated from a cross of ‘FL00-45’ (female parent, unpatented) by ‘FL97-130’ (pollen parent, unpatented). The new blueberry plant variety ‘FF04-14’ has been planted in replicated trials since 2008.

The new variety can be compared to commercial variety ‘Springhigh’ (U.S. Plant Pat. No. 16,404) in that ‘FF04-14’ has larger fruit that ripens later.

The new variety can be compared to commercial variety ‘Jewel’ (U.S. Plant Pat. No. 11,807) in that ‘FF04-14’ has larger, firmer fruit that ripens slightly later.

The new blueberry plant variety ‘FF04-14’ has maintained its distinguishing characteristics throughout successive asexual propagations. The variety has been repeatedly asexually reproduced through softwood cuttings in Lowell, Oreg., and the clones are phenotypically identical to the original plant.

### SUMMARY OF THE INVENTION

The new blueberry plant variety ‘FF04-14’ has not been observed under all possible environmental conditions. The phenotype may vary somewhat with variations in environment and cultural practices such as temperature and light intensity without, however, any variance in genotype.

The following traits have been repeatedly observed in Lowell, Oreg., and are determined to be the unique characteristics of the new blueberry plant variety ‘FF04-14’:
1. Vigorous plant with upright/round stature.
2. Large fruit
3. High yields

### BRIEF DESCRIPTION OF THE DRAWINGS

The accompanying photographic illustration shows typical specimens in full color of the foliage and fruit of the new variety ‘FF04-14’. The colors are as nearly true as is reasonably possible in a color representation of this type.

FIG. 1 is a photograph of the new variety ‘FF04-14’, taken Aug. 9, 2012; near Lowell, Oreg.

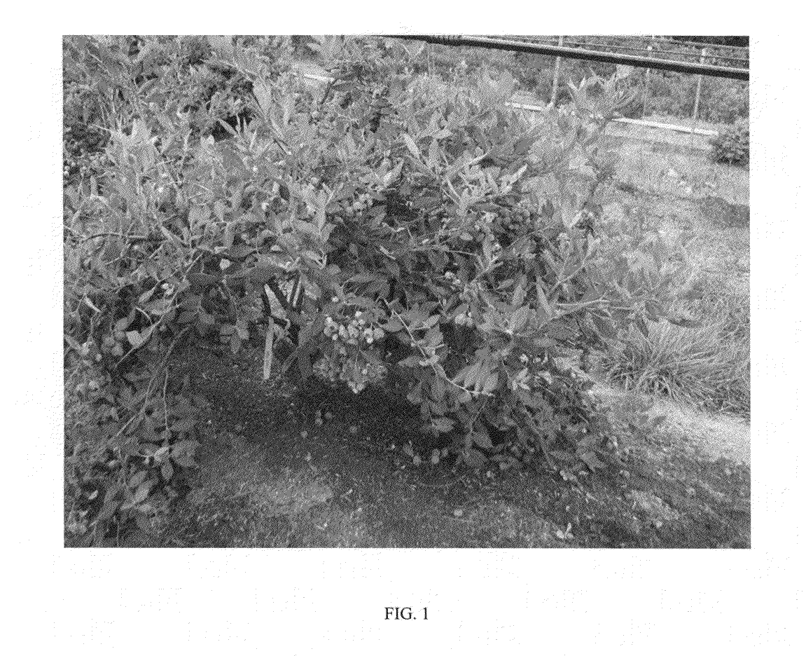

FIG. 2 is a photograph of the upper and lower surface of fruit of the new variety ‘FF04-14’ taken Jul. 13, 2012; near Lowell, Oreg.

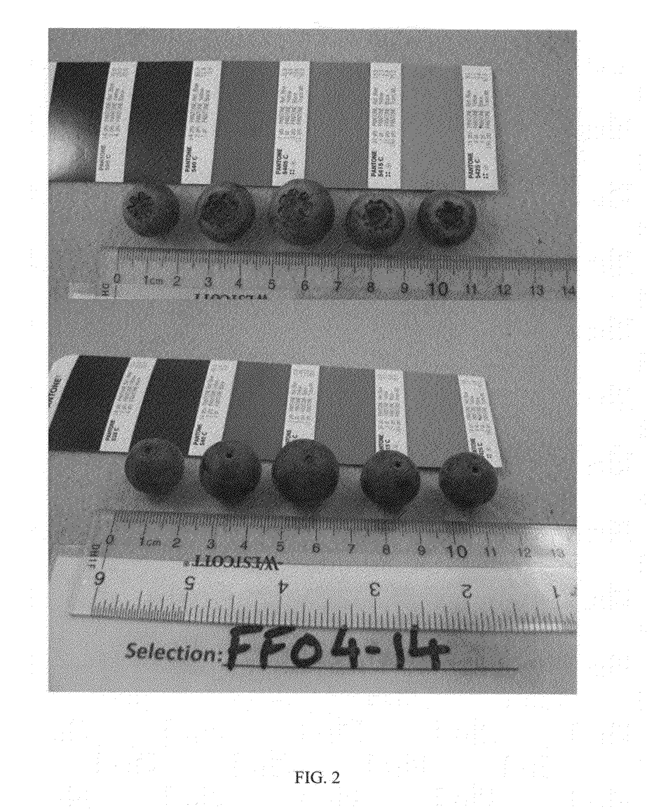

FIG. 3 is a photograph of the flowers of the new variety ‘FF04-14’, taken May 5, 2012; near Lowell, Oreg.

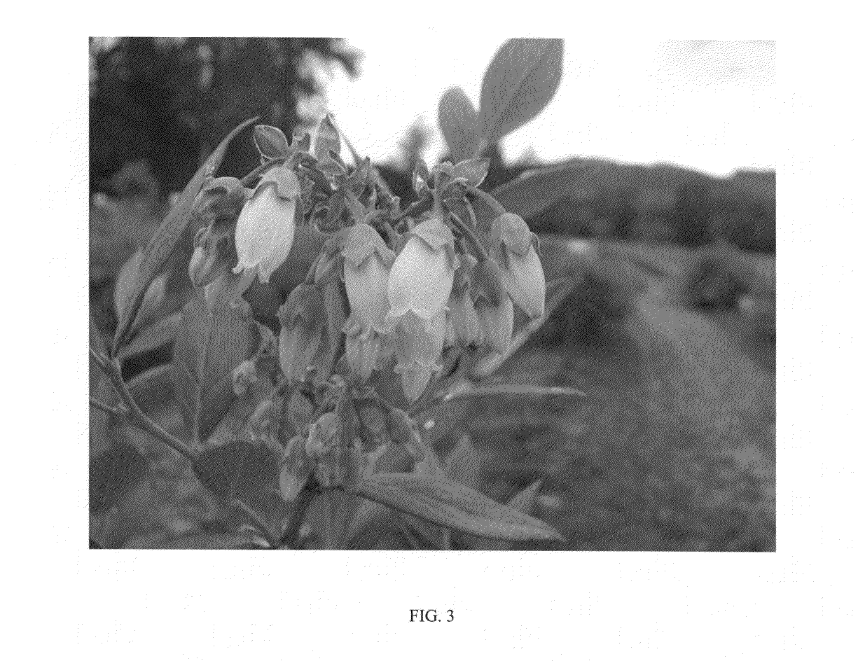

FIG. 4 is a photograph of the abaxial and adaxial sides of leaves of the new variety ‘FF04-14’, taken Jul. 13, 2012; near Lowell, Oreg.

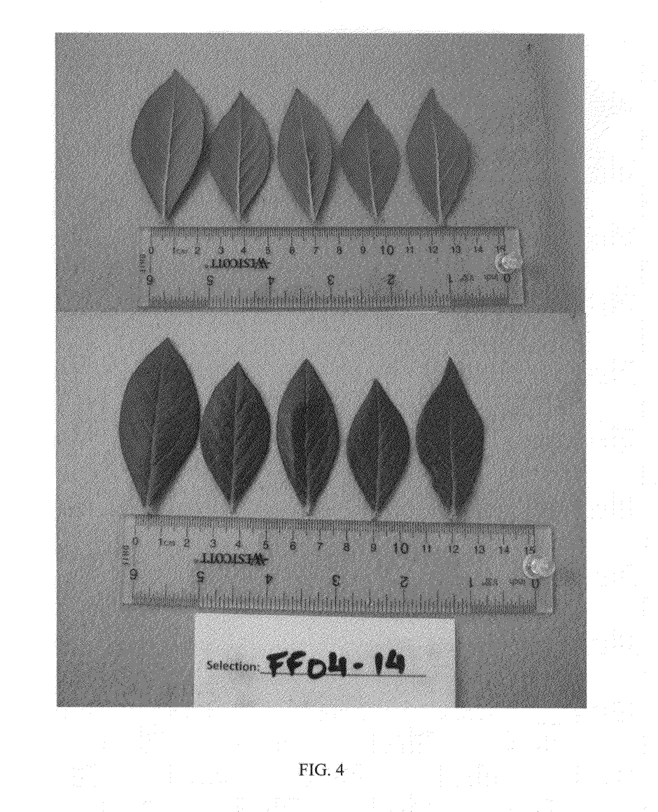

### BOTANICAL DESCRIPTION

The following detailed description sets forth the distinctive characteristics of ‘FF04-14’. The data which defines these characteristics was collected from asexual reproductions of the original selection. Dimensions, sizes, colors and other characteristics are approximations and averages set forth as accurately as possible. The plant history was taken on plants approximately 5 years of age and the descriptions relate to plants grown in the field in Lowell, Oreg. Plants of ‘FF04-14’ have also been observed near Delano, Calif., Tala, Jalisco, Mexico, and Colima City, Colima, Mexico. Where descriptions differ significantly between Lowell, Oreg. and other locations, reference has also been made to 2 year old plants in Colima, MX, and 3 year old plants in Delano, Calif. Color designations are from ‘The Pantone Book of Color” (by Leatrice Eiseman and Lawrence Herbert, Harry N. Abrams, Inc., Publishers, New York, 1990). Where the Pantone color designations differ from the colors in the photographs, the Pantone colors are accurate.
Classification:
   a. Family.—Ericaceae.
   b. Genus.—Vaccinium.
   c. Species.—corymbosum hybrid.
   d. Common Name.—Southern Highbush Blueberry.
Parentage: Female parent — FL00-45 (unpatented). Male parent — FL97-130 (unpatented).
Market Class: ‘FF04-14’ is a commercial variety intended for the hand harvest fresh market.
Plant:
Plant size:
   a. Height of plant.—Average 43″.
   b. Canopy diameter.—Average 34″.
Growth habit: Upright, round
Plant vigor: Vigorous
Twigginess: Low
Suckering: Low
Evergreenness: Evergreen in Oreg.
Chilling requirement: Approximately 300-500 hours
Cold hardiness: Approximately USDA hardiness zones 7-9
Productivity: In Colima, Mexico on one year old plants average 1.43 kilos per plant. In Delano, Calif. on 4 year old plants average 7 lbs per plant.
Propagation: Easy to propagate from softwood cuttings
Disease resistance/susceptibility:
   a. Root rot (Phytophthora cinnamomi).—Does not appear to be susceptible.
   b. Stem blight (Botryosphaeria spp).—Does not appear to be susceptible.
   c. Phomopsis twig blight (Phomopsis vaccinii).—Does not appear to be susceptible.
   d. Botryosphaeria stem canker (Botryosphaeria corticis).—Does not appear to be susceptible.
   e. Bacterial cane canker (Pseudomonas syringae).—Does not appear to be susceptible.
   f. Botrytis blight (Botrytis cinerea).—Does not appear to be susceptible.
   g. Leaf spot (Septoria spp.).—Does not appear to be susceptible.
   h. Leaf rust (Naohidemyces vaccinit).—Does not appear to be susceptible.
Survival: Good
Wood:
Texture:
   a. Surface texture new wood.—Smooth.
   b. Surface texture 1 year wood.—Smooth turning rough.
   c. Surface texture 3 year wood.—Rough.
Color:
   a. Color new wood.—Pantone color Green Banana 14-0434.
   b. Color bark 1 year.—Pantone colors Green Oasis 15-0538 and Mahogany 18-1425.
   c. Color bark 3 year.—Pantone colors Shell 13-1405, Pinecone 19-1121 and Camel 17-1224.
Internode length: Average 20.95 mm
Dimensions:
   a. Leaf length.—Average 60.82 mm.
   b. Leaf width.—Average 27.15 mm.
Leaf shape: Elliptical with acute apex and base
Leaf margin: Slightly crenulate
Color:
   a. Leaf color upper leaf surface.—Pantone color Black Forest 19-0315.
   b. Leaf color lower leaf surface.—Pantone color Water Cress 17-0220.
Pubescence:
   a. Pubescence upper leaf surface.—Absent.
   b. Pubescence lower leaf surface.—Absent.
   c. Pubescence leaf margins.—Absent.
Petiole:
   a. Petiole length.—Average 1.69 mm.
   b. Petiole color.—Pantone color Mellow Green 12-0426.
   c. Petiole surface texture.—Smooth.
Leafing vs. flowering: Flower bud break and vegetative bud break occur at approximately the same time in Lowell, Oreg. Flowers persist through initial vegetative growth period.
Flowering arrangement: Alternate
Immature flower color: Pantone colors Pastel Parchment 11-0603 to Pastel Yellow 11-0616 and Hollyberry 17-1633
Fragrance: Sweet
Flower shape: Cylindrical
Flower length: Average 12.97 mm.
Pedicel:
   a. Pedicel length.—Average 10.9 mm.
   b. Pedicel color.—Pantone color Green Olive 17-0535.
Peduncle:
   a. Peduncle length.—Average 14.87 mm.
   b. Peduncle color.—Pantone colors Mellow Green 12-0426 and Green Oasis 15-0538.
Pollen:
   a. Pollen staining.—Staining with acetocarmine: approximately 90%.
   b. Pollen abundance.—Moderate.
   c. Pollen color.—Pantone color Pale Banana 12-0824.
Corolla:
   a. Corolla length.—Average 9.12 mm.
   b. Corolla diameter.—Average 7.2 mm.
   c. Corolla aperture.—Average 4.65 mm.
   d. Corolla color.—Pantone color Pastel Parchment 11-0603.
   e. Corolla texture.—Smooth, with ridges at petal junctures.
Style Length: Average 7.42 mm.
Calyx:
   a. Calyx diameter.—Average 5.92 mm.
   b. Calyx surface texture.—Smooth.
   c. Calyx color.—Pantone colors Green Olive 17-0535, Peridot 17-0336 and Holly Berry 17-1633 on tips of some sepals.
Pistil color: Pantone color Green Oasis 15-0538
Flowering cluster: Loose
Flowering period: In Lowell, Oreg., flowers from mid to late April through late May
Number flowers: Average 6-7 in Lowell, Oreg.
Stigma location: Average 1.07 mm below lip of corolla
Anther:
   a. Anther length.—Average 2.74 mm.
   b. Anther color.—Pantone colors Autumn Leaf 17-1347 and Sierra 18-1239.
Self-compatibility: Moderate. Approximately 55% of self-pollinated flowers reach maturity.
Berries:
Mean harvest date: Begins to ripen around 15 January in Colima, MX and 5 May near Delano, Calif. Not very productive in Oreg.
Mean date last pick: Typically harvest ends by 30 April in Colima, MX and approximately 5 June near Delano, Calif.
Quantity berries per cluster: Average of 6 in Oreg. and 9 in Colima, MX
Calyx:
   a. Calyx aperture.—Average 7.22 mm.
   b. Calyx lobes.—5 moderately distinct lobes.
   c. Calyx depth.—Average 0.82 mm in Oreg. Very shallow in Oreg., moderately shallow in Central Mexico.
Berry cluster: Loose
Berry pedicel:
   a. Length.—Average 9.93 mm
   b. Surface texture.—Smooth
Berry peduncle:
   a. Length.—Average 18.89 mm
   b. Surface texture.—Smooth
Berry detachment: Moderately easy
Berry weight: Average 2.93 g in Oreg.
Berry height: Average 15.41 mm in Oreg.
Berry width: Average 18.54 mm in Oreg.
Berry shape: Oblate
Berry skin color:
   a. Immature berry color.—Pantone colors Leaf Green 15-0332, Piquant Green 17-0235 and some Crimson 19-1762 on calyx.
   b. Berry skin color on plant.—Pantone color Dapple Gray 16-3907.
   c. Berry skin color after harvest.—Pantone colors Dapple Gray 16-3907 and India Ink 19-4019.
   d. Berry skin color after polishing.—Pantone color Dark Navy 19-4013.
Berry wax: Moderate wax and moderately persistent
Berry flesh color: Pantone color Citron 12-0524
Pedicel scar: Moderate in size, average 1.59 mm in Oreg. Scar is larger on fruit from current season's growth in Colima, MX.
Berry firmness: Medium in Lowell Oreg., good to excellent in Colima, MX.
Berry flavor: Sweet, low acidity
Berry texture: Smooth
Seed:
Seed color: Pantone color Bran 17-1336
Seed length: Average 1.66 mm
Seed width: Average 1.02 mm
Seed weight: Average 0.5 mg
Quantity seeds per berry: Average 17 in Oreg.

1. A new and distinct variety of blueberry plant named ‘FF04-14,’ substantially as illustrated and described herein.
